(19) United States
(12) Patent Application Publication
(10) Pub. No.: US 2021/0398640 A1
(43) Pub. Date: Dec. 23, 2021

(54) ACTIVITY EVALUATIONS AND USER ADHERENCE FOR DEVICES

(71) Applicant: Amazon Technologies, Inc., Seattle, WA (US)

(72) Inventors: Herman Singh Dhak, Seattle, WA (US); Beshoy Sarkis, Bothell, WA (US); Maulik Majmudar, Medina, WA (US); Andreas Caduff, Clyde Hill, WA (US)

(73) Assignee: Amazon Technologies, Inc., Seattle, WA (US)

(21) Appl. No.: 16/904,318

(22) Filed: Jun. 17, 2020

(51) Int. Cl. G16H 20/30; G16H 40/60

(52) U.S. Cl. CPC G16H 20/30; G16H 40/60

(57) ABSTRACT

Devices, systems, and methods are provided for performing activity evaluations. A method may include determining, by a device, a heart rate. The method may include determining, based on an activity template, a first biometric and a threshold associated with the first biometric. The method may include determining first data associated with the first biometric, the first data indicative of a first quantity. The method may include comparing, by the at least one processor, the first quantity to the threshold, and determining, based on the first data, second data associated with a second biometric, the first biometric different than the second biometric, the second data indicative of a second quantity. The method may include determining that the first quantity is associated with the second quantity. The method may include sending a message to a second device for presentation, the message associated with the first biometric.

### BACKGROUND

People increasingly are monitoring their activities and consumption habits to improve their health. Some activities that people may monitor include exercise, rest, and sedentary periods. People may be interested in the amount of time that they spend performing certain activities. However, some activity tracking methods using devices do not consider the effects that some activity of a person have on other activity of the person, and do not allow a person to track goals with multiple criteria. Therefore, people may benefit from an enhanced activity evaluation and user adherence using devices.

### BRIEF DESCRIPTION OF THE DRAWINGS

FIG. 1 illustrates an example system for activity evaluations using devices, in accordance with one or more example embodiments of the present disclosure.

FIG. 2 illustrates an example system for activity evaluations using devices, in accordance with one or more example embodiments of the present disclosure.

FIG. 3 illustrates an example flow diagram for performing activity evaluations, in accordance with one or more example embodiments of the present disclosure.

FIG. 4 illustrates an example flow diagram for performing activity evaluations, in accordance with one or more example embodiments of the present disclosure.

FIG. 5 illustrates a flow diagram for a process for performing activity evaluations, in accordance with one or more example embodiments of the present disclosure.

FIG. 6 illustrates a block diagram of an example machine upon which any of one or more techniques (e.g., methods) may be performed, in accordance with one or more example embodiments of the present disclosure.

Certain implementations will now be described more fully below with reference to the accompanying drawings, in which various implementations and/or aspects are shown. However, various aspects may be implemented in many different forms and should not be construed as limited to the implementations set forth herein; rather, these implementations are provided so that this disclosure will be thorough and complete, and will fully convey the scope of the disclosure to those skilled in the art. Like numbers in the figures refer to like elements throughout. Hence, if a feature is used across several drawings, the number used to identify the feature in the drawing where the feature first appeared will be used in later drawings.

### DETAILED DESCRIPTION

### Overview

Example embodiments described herein provide certain systems, methods, and devices for performing activity evaluations and user adherence.

A person's activities may be evaluated in a variety of ways. For example, user device data, such accelerometer or other motion and/or location data, may provide an indication of a person's activity levels (e.g., whether the person with the user device moved a certain amount during a time period). Biometric data, such as heart rate (HR), breathing rate, pulse oximetry, body fat, hydration level, body temperature, blood sugar, and the like, may indicate whether a person is sleeping, sedentary, or active. The combination of device and biometric data may provide indications of activity levels of a person over a period of time, such as a day or a week. Some activity monitoring techniques may not consider the effects on some biometric data on other biometric data, and may not track whether users are meeting activity and/or biometric goals that depend on multiple data types.

For example, some devices may determine whether a person met an activity goal one day, but not whether the activity goal was met multiple days over a longer period of time (e.g., whether a person slept a threshold number of hours per night during a week or month, whether a person exercised a threshold number of days during a week or month, etc.). Some devices also may lack the ability to indicate to a user whether the user is on pace to meet an activity goal, and to indicate how much of an activity a person may need over one or multiple days to achieve the goal based on real-time and/or previous day data. Some devices may lack the ability to determine whether a user is meeting a goal that considers a combination of thresholds for different types of data (e.g., did the user sleep a threshold number of hours for a threshold number of nights and/or exercise a threshold amount of time for a threshold number of days during a week, etc.).

Some devices may not compare the effects of one type of biometric and/or activity data on another type of biometric and/or activity data. For example, some devices may not consider a relationship between the amount of time that a person slept or exercised and the person's heart rate, how much time that a person slept and the person's running pace, the amount of time that a person exercised and the person's heart rate or breathing rate, etc.

Therefore, people may benefit from an enhanced method of using a person's activity and biometric data to monitor multiple activity goals and to determine relationships between different types of activity and/or biometric data.

In one or more embodiments, a computer-executable service may include experiments that define instructions for users to follow to improve their wellness. A computer-executable service may, with user consent and in compliance with any relevant laws, determine whether a user is adhering to the instructions, and may notify a user regarding whether the user is adhering to the instructions. Determination of whether a user adhered to instructions (e.g., goals) may be based on data input by the user (e.g., the user may input data using a device, the data indicating biometric and/or activity data, such as that the user exercised, slept, etc. for a period of time) and/or data detected automatically (e.g., with user consent and in compliance with any relevant laws) by the device (e.g., using one or more sensors detecting biometric data of the user and/or motion associated with a device). In this manner, the computer-executable service may determine whether a user adheres to an activity goal without requiring the user to self-report activity and/or biometric data.

In one or more embodiments, with user consent and in compliance with any relevant laws, a device or system may store multiple types of data (e.g., using data stores or other data storage), such as heart rate data, breathing rate data, walking data, running data, sleeping data, eating data, drinking data, and the like. Users may set activity goals that correspond to one or more types of data, such as running for thirty minutes (e.g., a threshold amount of time) three times a week (e.g., a first threshold number of times) for at least three weeks in a month (e.g., a second threshold number of times). In this manner, the device may track segments of activity (e.g., did the user achieve a threshold number of segments of activity satisfying a threshold amount of time).

In one or more embodiments, the device may, based on the goals, identify the types of data to monitor, and may retrieve the relevant data from the data storage for analysis. For example, a device (e.g., a smartphone, wearable device, computer device, etc.) may query a network (e.g., a cloud-based network) for one or more types of data, such as the number of running sessions, walking sessions, sleeping sessions, consumption sessions, etc. during a week. The query may specify the time range(s) for which the data is desired (e.g., particular days, weeks, months, hours, etc.), and may filter data returned in response to the query based on the time range(s).

In one or more embodiments, templates (e.g., biometric data templates) may define the thresholds used to monitor a person's adherence to one or more goals. Adherence rules may be defined by a template. For example, an adherence rule may refer to a biometric and its corresponding threshold(s) and/or rules, which may be evaluated by a string that defines the order in which the device may evaluate the rules using Boolean operators (e.g., and, or, etc.). For example, a template may define a rule that a person must exercise a certain amount of time for a threshold number of segments in a week, and/or must sleep a threshold amount of time for a threshold number of segments in a week. The order in which the device may evaluate the relevant data to determine whether a person met a goal may be defined by the string (e.g., a computer function). In an example in which the goal is to run for at least thirty minutes and sleep between 7-9 hours every night for a week, the adherence rules may include Rule 1: A metric greater than or equal to a threshold of thirty minutes using a biometric “total running duration,” and Rule 2: A metric between thresholds of seven and nine hours of sleep using the biometric “sleep duration.” The device may fetch (e.g., request using a request call, such as a JSON) the metrics individually by querying the network biometric data storage, may receive the queried data, and may determine Boolean results (e.g., true or false) against the thresholds (e.g., true or false that a user ran for at least thirty minutes, true or false that the user slept 7-9 hours every night for a week). An aggregated rule expression may rely on one or more of the Boolean results (e.g., true and true=true—user adhered to the goal; true and false=false; user did not adhere to the goal) Likewise, the network may receive queries identifying data types, and may provide the data types to the device.

In one or more embodiments, a user's adherence may be based on units of time during which the user's adherence may be tracked (e.g., a day beginning at midnight and ending at 11:59 PM). An adherence segment may refer to a collection of adherence units of time (e.g., a segment may refer to one day or a block of hours). An adherence segment may have its own adherence value based on respective adherences of individual time units. An adherence metric may refer to a biometric used to determine a customer's adherence (e.g., a step count, walking/running sessions, sleeping sessions, etc.). An adherence rule may be used to determine whether an adherence metric should count toward determining a user's adherence (e.g., whether an adherence metric was greater than a threshold, less than a threshold, between thresholds, etc.). A rule output may refer to a result of an adherence determination based on an adherence rule, and may include an indication of what the target adherence metric value was and what the user's actual value was for the adherence metric. For example, an experiment may set a goal of a user having less than eight hours of sedentary time for four weeks. The query and metric may include an aggregation query of “none” or “zero” for an activity intensity metric. The time unit rule used to evaluate the metric may be a rule of less than or equal to a threshold (e.g., zero or none) for any time units (e.g., any given day), and the segment rule may be a number of days (e.g., days during which the time unit rule was adhered to) satisfying a threshold of 28 days (e.g., four weeks). Another experiment may be to job for thirty minutes three times in a week or to run at least three times in a week. The query and metric may include a run workout session query to receive running session data. The time unit rule used to evaluate the metric may be a rule of based on the OR Boolean condition of the query, and the segment rule may be a number of days adhered (e.g., a number of days when the jogging or running was adhered to) satisfying a threshold of three days.

In one or more embodiments, the device may determine whether the adherence or failure to adhere to a goal may correlate to an effect on a user's biometric data and/or the user's adherence or failure to adhere to another goal. For example, the device may determine whether a person adhering to an exercise goal correlates to whether the person slept and/or ate a sufficient amount, whether a user exercising affected the user's heart rate, whether the user running a number of times affected the user's running pace, and the like. The relationships between data may be evaluated for real-time, past, and future behavioral determinations, such as whether a person may sleep better if the person adhered to an exercise goal, etc. In this manner, the device may identify the impact of some biometric data on other biometric data, and may indicate the impact to the user to allow the user to make behavioral decisions. The device may provide notifications and instructions to the user (e.g., sleep more, exercise more, go to bed earlier, etc.), and/or to other devices (e.g., turn off the television or stereo at a certain time, change a room temperature, etc.).

The above descriptions are for purposes of illustration and are not meant to be limiting. Numerous other examples, configurations, processes, etc., may exist, some of which are described in greater detail below. Example embodiments will now be described with reference to the accompanying figures.

### Illustrative Processes and Use Cases

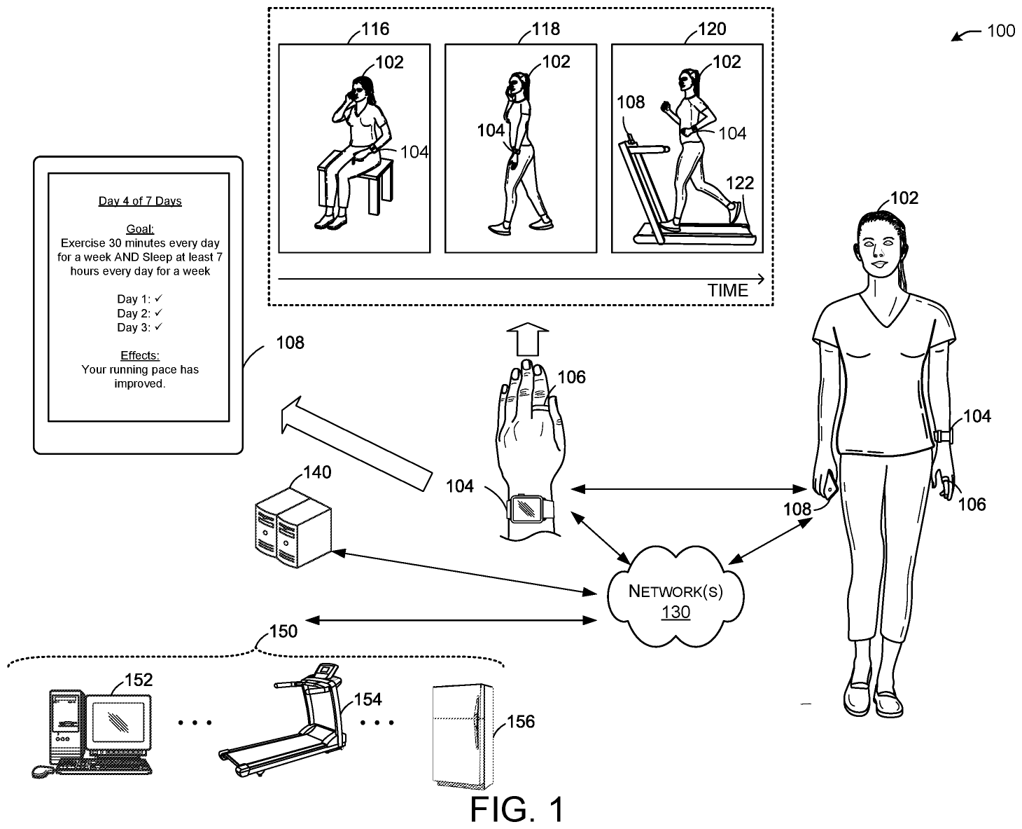

FIG. 1 illustrates an example system 100 for activity evaluations using devices, in accordance with one or more example embodiments of the present disclosure.

Referring to FIG. 1, the system 100 may include a user 102 with multiple devices (e.g., device 104, device 106, device 108). For example, the user 102 may be wearing the device 104 (e.g., a wrist watch) and the device 106 (e.g., a ring device), and may be holding or carrying the device 108 (e.g., a smartphone). At step 116 (e.g., a time), the user 102 may be sedentary (e.g., sitting). At step 118 (e.g., a time), the user 102 may be walking (e.g., exercising lightly or moderately). At step 120, the user 102 may be jogging or running on a treadmill 122 (e.g., exercising moderately or vigorously). Step 116, step 118, and step 120 may represent different times throughout a day or multiple days (e.g., a week, month, etc.). The user 102 may be wearing or holding any one or more of the device 104, the device 106, and/or the device 108 at any of step 116, step 118, and step 120, or any one or more of the device 104, the device 106, and/or the device 108 may be otherwise monitoring, with user consent and consistent with appropriate laws, activity of the user 102 as explained further herein.

Still referring to FIG. 1, the system 100 may include one or more servers 140 (e.g., cloud-based servers remote from the device 104, the device 106, and/or the device 108), which may receive data from any one or more of the device 104, the device 106, and/or the device 108 (e.g., corresponding to step 116, step 118, and/or step 120). The data received by the one or more servers 140 from any one or more of the device 104, the device 106, and/or the device 108 may include biometric data. The one or more servers 140 may analyze the biometric data to determine amounts of activities (e.g., walking, running, eating, sleeping, etc.) performed by the user 102 over a period of time (e.g., a week, a month, etc.). The one or more servers 140 may determine the amounts of time that the user 102 exercised and/or spent sedentary. The one or more servers 140 may determine the total and average number of steps (e.g., a daily or weekly total or average) that the user 102 performed over a time period. The one or more servers 140 may determine, using the biometric data, the user's heart rate, breathing rate, body fat, hydration levels, body temperature, blood glucose levels, and the like corresponding to the times when a person was active, sedentary, consuming food or liquid, and the like. Alternatively, any of the device 104, the device 106, and/or the device 108 may collect the device and/or biometric data, and may perform the evaluations for amounts of activity using biometric data. The one or more servers 140 and/or any of the device 104, the device 106, and/or the device 108 may determine provide activity information (e.g., including activity levels and whether the user 102 has satisfied activity goals) to any of the device 104, the device 106, and/or the device 108 for presentation.

Still referring to FIG. 1, the one or more servers 140 may communicate (e.g., using one or more communication networks 130) with one or more devices 150 (e.g., computer device 152, treadmill 154, refrigerator 156) using the one or more communication networks 130 or using a direct connection (e.g., Wi-Fi, Bluetooth, ultrasound). The one or more servers 140 may receive data captured by the device 104, the device 106, the device 108, and/or the one or more devices 150 and may analyze the data. With user consent, the one or more servers 140 may provide user data, such as health data, data regarding the user's product consumption habits and history, exercise and other activity data, and the like. The one or more devices 150 may provide data indicating when a user exercised or bought consumable products (e.g., using browsing or other search history from the computer device 152, or medical data such as medical history or prescription product history from the computer device 152). Such data from the one or more devices 150 may indicate activity options (e.g., exercising options available to a user) and for analysis regarding whether a user is exercising after consuming certain types of products. The one or more servers 140 may send messages to control operation of the one or more devices 150 to help the user 102 achieve a goal, such as to adjust room temperature to facilitate sleeping or exercising, to control lighting, to display messages encouraging the user 102 to exercise, rest, consume food or liquid, or not consume food or liquid. The one or more devices 150 may include smart devices, Internet of things (IoT) devices, and the like.

In one or more embodiments, the one or more servers 140 may have access to experiments that define instructions for users to follow to improve their wellness. The one or more servers 140 may, with user consent and in compliance with any relevant laws, determine whether the user 102 is adhering to the instructions, and may notify the user 102 regarding whether the user 102 is adhering to the instructions.

In one or more embodiments, with user consent and in compliance with any relevant laws, the one or more servers 140 may store or have access to multiple types of data (e.g., using data stores or other data storage), such as heart rate data, breathing rate data, walking data, running data, sleeping data, eating data, drinking data, and the like. The user 102 may set activity goals that correspond to one or more types of data, such as running for a threshold amount of time three times a week (e.g., a first threshold number of times) for at least three weeks in a month (e.g., a second threshold number of times). In this manner, the one or more servers 140 may track segments of activity (e.g., did the user achieve a threshold number of segments of activity satisfying a threshold amount of time).

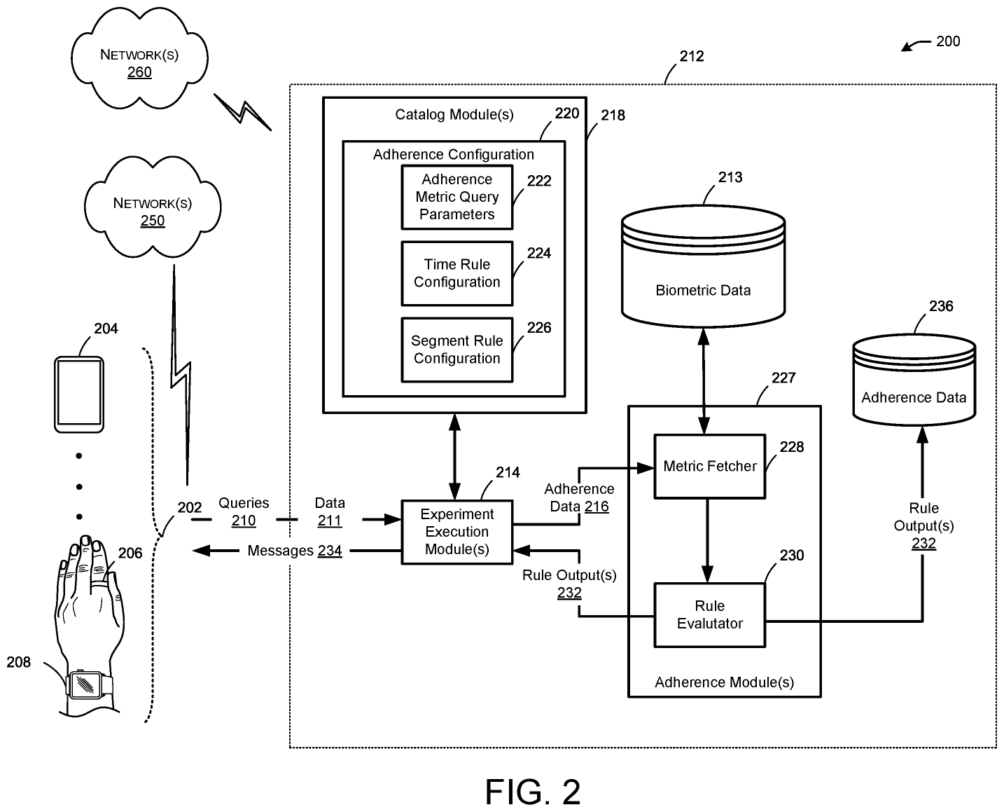

In one or more embodiments, the one or more servers 140 may, based on the goals and activity templates (e.g., biometric data templates), identify the types of data to monitor. For example, one or more servers 140 may query a service (as explained further in FIG. 2) for one or more types of data, such as the number of running sessions, walking sessions, sleeping sessions, consumption sessions, etc. during a week. The query may specify the time range(s) for which the data is desired (e.g., particular days, weeks, months, hours, etc.), and may filter data returned in response to the query based on the time range(s).

In one or more embodiments, templates may define the thresholds used to monitor a person's adherence to one or more goals. Adherence rules may be defined by a template. For example, and adherence rule may refer to a biometric and its corresponding threshold(s) and/or rules, which may be evaluated by a string that defines the order in which the device may evaluate the rules using Boolean operators (e.g., and, or, etc.). For example, a template may define a rule that the user 102 must exercise a certain amount of time for a threshold number of segments in a week, and/or must sleep a threshold amount of time for a threshold number of segments in a week. The order in which the device may evaluate the relevant data to determine whether the user 102 met a goal may be defined by the string (e.g., a computer function). In an example in which the goal is to run for at least thirty minutes and sleep between 7-9 hours every night for a week, the adherence rules may include Rule 1: A metric greater than or equal to a threshold of thirty minutes using a biometric “total running duration,” and Rule 2: A metric between thresholds of seven and nine hours of sleep using the biometric “sleep duration.” The device may fetch (e.g., request using a request call, such as a JSON) the metrics individually by querying the network biometric data storage, may receive the queried data, and may determine Boolean results (e.g., true or false) against the thresholds (e.g., true or false that a user ran for at least thirty minutes, true or false that the user slept 7-9 hours every night for a week). An aggregated rule expression may rely on one or more of the Boolean results (e.g., true and true=true—user adhered to the goal; true and false=false; user did not adhere to the goal). Likewise, the network may receive queries identifying data types, and may provide the data types to the device.

In one or more embodiments, a user's adherence may be based on units of time during which the user's adherence may be tracked (e.g., a day beginning at midnight and ending at 11:59 PM). An adherence segment may refer to a collection of adherence units of time (e.g., a segment may refer to one day or a block of hours). An adherence segment may have its own adherence value based on respective adherences of individual time units. An adherence metric may refer to a biometric used to determine the user's adherence (e.g., a step count, walking/running sessions, sleeping sessions, etc.). An adherence rule may be used to determine whether an adherence metric should count toward determining a user's adherence (e.g., whether an adherence metric was greater than a threshold, less than a threshold, between thresholds, etc.). A rule output may refer to a result of an adherence determination based on an adherence rule, and may include an indication of what the target adherence metric value was and what the user's actual value was for the adherence metric. For example, an experiment may set a goal of the user 102 having less than eight hours of sedentary time for four weeks. The query and metric may include an aggregation query of “none” or “zero” for an activity intensity metric. The time unit rule used to evaluate the metric may be a rule of less than or equal to a threshold (e.g., zero or none) for any time units (e.g., any given day), and the segment rule may be a number of days (e.g., days during which the time unit rule was adhered to) satisfying a threshold of 28 days (e.g., four weeks). Another experiment may be to job for thirty minutes three times in a week or to run at least three times in a week. The query and metric may include a run workout session query to receive running session data. The time unit rule used to evaluate the metric may be a rule of based on the OR Boolean condition of the query, and the segment rule may be a number of days adhered (e.g., a number of days when the jogging or running was adhered to) satisfying a threshold of three days.

In one or more embodiments, the one or more servers 140 may determine whether the adherence or failure to adhere to a goal may correlate to an effect on the user's biometric data and/or the user's adherence or failure to adhere to another goal. For example, the one or more servers 140 may determine whether the user 102 adhering to an exercise goal correlates to whether the user 102 slept and/or ate a sufficient amount, whether the user 102 exercising affected the user's heart rate, whether the user running a number of times affected the user's running pace, and the like. The relationships between data may be evaluated for real-time, past, and future behavioral determinations, such as whether the user 102 may sleep better if the person adhered to an exercise goal, etc. In this manner, the one or more servers 140 may identify the impact of some biometric data on other biometric data, and may indicate the impact to the user 102 to allow the user 102 to make behavioral decisions. The one or more servers 140 may provide notifications and instructions to the device 104, the device 106, and/or the device 108 (e.g., sleep more, exercise more, go to bed earlier, etc.), and/or to other devices (e.g., turn off the television or stereo at a certain time, change a room temperature, etc.). Determining user adherence to a goal may be based on user inputs indicating user activity and/or biometric data, and/or based on an automatic determination by the one or more servers 140 (e.g., using data collected by the one or more servers 140 by any of the other devices) using data indicative of user activity and/or user biometric data.

In one or more embodiments, as shown in FIG. 1, the messages sent to a device presentation may indicate goals, outcomes, effects/relationships/associations, recommendations, and the like. For example, a goal may be to exercise for thirty minutes every day for a week and sleep at least seven hours every day for a week. For the goal to be satisfied, the outcomes of both exercising thirty minutes for seven consecutive days and sleeping at least seven hours for seven consecutive nights must be true. The outcome may be presented to the user 102 (e.g., using the device 104, the device 106, the device 108, etc.) to indicate whether the goal was satisfied/achieved and/or is on pace to be achieved. The one or more servers 140, the device 104, the device 106, and/or the device 108 may present effects, such as indications that exercising helps the user 102 sleep and/or that sleeping helps the user 102 exercise. The one or more servers also may evaluate biometric data to determine whether satisfying or failing to satisfy a goal corresponds to any changes in other biometric data. For example, when the user 102 satisfies the goal of running for thirty minutes every day for a week, the one or more servers 140 may use the satisfaction of the running goal to evaluate changes or differences in other data, such as running time or pace. When running data indicates that the user 102 improved a running pace during the week that the user 102 satisfied the goal of running every day for thirty minutes (e.g., by comparing amounts of activity indicated by the data over multiple times), the one or more servers 140 may determine a correlation/association/effect, such as satisfying the running goal may have improved the running pace of the user 102.

In one or more embodiments, the messages sent to a device presentation may indicate, in real-time, the user's progress/adherence to goals. For example, when segment goals exist (e.g., adhering to a goal for a respective day multiple days during a time period), the messages may indicate whether the user 102 has adhered to or not adhered to a goal in respective segments (e.g., whether the user adhered to goal criteria for any day during a week), and/or whether the user 102 is on pace to adhere to a goal (e.g., has the user exercised enough today, slept enough today, etc.).

In one or more embodiments, the device 104, the device 106, the device 108, and/or the one or more servers 140 may include a personal computer (PC), a smart home device, a wearable wireless device (e.g., bracelet, watch, glasses, ring, etc.), a desktop computer, a mobile computer, a laptop computer, an Ultrabook™ computer, a notebook computer, a tablet computer, a server computer, a handheld computer, a handheld device, an internet of things (IoT) device, a sensor device, a PDA device, a handheld PDA device, an on-board device, an off-board device, a hybrid device (e.g., combining cellular phone functionalities with PDA device functionalities), a consumer device, a vehicular device, a non-vehicular device, a mobile or portable device, a non-mobile or non-portable device, a mobile phone, a cellular telephone, a PCS device, a PDA device which incorporates a wireless communication device, a mobile or portable GPS device, a DVB device, a relatively small computing device, a non-desktop computer, a “carry small live large” (CSLL) device, an ultra mobile device (UMD), an ultra mobile PC (UMPC), a mobile internet device (MID), an “origami” device or computing device, a device that supports dynamically composable computing (DCC), a context-aware device, a video device, an audio device, an A/V device, a set-top-box (STB), a Blu-ray disc (BD) player, a BD recorder, a digital video disc (DVD) player, a high definition (HD) DVD player, a DVD recorder, a HD DVD recorder, a personal video recorder (PVR), a broadcast HD receiver, a video source, an audio source, a video sink, an audio sink, a stereo tuner, a broadcast radio receiver, a flat panel display, a personal media player (PMP), a digital video camera (DVC), a digital audio player, a speaker, an audio receiver, an audio amplifier, a gaming device, a data source, a data sink, a digital still camera (DSC), a media player, a smartphone, a television, a music player, or the like. Other devices, including smart devices such as lamps, climate control, car components, household components, appliances, etc. may also be included in this list.

The device 104, the device 106, the device 108, and/or the one or more servers 140 may be configured to communicate via a communications network 130, wirelessly or wired (e.g., the same or different wireless communications networks). The communications network 130 may include, but not limited to, any one of a combination of different types of suitable communications networks such as, for example, broadcasting networks, cable networks, public networks (e.g., the Internet), private networks, wireless networks, cellular networks, or any other suitable private and/or public networks. Further, communications network 130 may have any suitable communication range associated therewith and may include, for example, global networks (e.g., the Internet), metropolitan area networks (MANs), wide area networks (WANs), local area networks (LANs), or personal area networks (PANs). In addition, communications network 130 may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, white space communication mediums, ultra-high frequency communication mediums, satellite communication mediums, or any combination thereof.

FIG. 2 illustrates an example system 200 for performing activity evaluations, in accordance with one or more example embodiments of the present disclosure.

Referring to FIG. 2, the system 200 may include one or more devices 202 (e.g., device 204, device 206, and device 208 similar to the device 104, the device 106, and the device 108 of FIG. 1) that may send queries 210 and data 211 (e.g., biometric data indicative of user activity) to a network 212 (e.g., a cloud-based network, similar to the one or more servers 140 of FIG. 1). One or more experiment execution modules 214 may receive the queries 210, and may obtain adherence data 216 from one or more catalog modules 218, which may include an adherence configuration 220, including adherence metric query parameters 222, a time rule configuration 224, and a segment rule configuration 226. The adherence metric query parameters 222 may include tracked biometric data (with user consent, such as step count, walk/run sessions, etc.). The time rule configuration 224 may indicate a threshold amount of time for which a user's activity is to satisfy a threshold (e.g., when a goal is to run for thirty minutes, the threshold may be thirty minutes). The segment rule configuration 226 may indicate a threshold number of days for which the threshold amount of time is to be satisfied. The one or more experiment execution modules 214 may receive the adherence data 216, including the adherence metric query parameters 222, from the one or more catalog modules 218, and may provide the adherence data 216 to one or more adherence modules 227 of the network 212. The one or more adherence modules 227 may include a metric fetcher 228 an a rule evaluator 230.

Still referring to FIG. 2, the metric fetcher 228 may send queries to one or more biometric data stores 230 to receive biometric data relevant to one or more goals defined by a biometric data template. The queries may include the adherence metric query parameters 222 from the one or more catalog modules 218 so that the biometric data returned to the metric fetcher 228 may include relevant data for tracking a user's adherence to the one or more goals defined by a biometric data template. For example, the queries to the one or more biometric data stores 231 may indicate that a biometric data template sets goals for biometric data such as walking, running, sleeping, eating, drinking, etc. The biometric data provided in response to the queries from the metric fetcher 228 may include data indicative of amounts of activities performed by a user, the amounts of activities indicated by the biometric data. For example, the biometric data may indicate the times and durations when a user was walking, running, sleeping, eating, drinking, etc. The metric fetcher may provide the biometric data to the rule evaluator 230 for analysis based on thresholds set by goals of a biometric data template. The rule evaluator 230 may determine whether the biometric data indicates that a user performed an amount of activity satisfying a threshold amount, how many times the user performed an amount of activity satisfying a threshold amount, and whether the number of times that user performed an amount of activity satisfying a threshold amount satisfies a threshold. For example, when a biometric data template indicates an activity goal of exercising at least one hour per day for at least three days a week and/or sleeping at least seven hours per night for a week, the rule evaluator 230 may use walking or running biometric data to determine whether the user exercised at least sixty minutes in a day for any day in the week, and whether the number of days when the user exercised at least sixty minutes exceeds a threshold number of days. The result of this analysis may be a “true” or “false” output. The rule evaluator 230 may use sleeping biometric data to determine whether the user slept at least seven hours a night for any nights during the week, and whether the number of days when the user slept at least seven hours a night exceeds a threshold number of nights. The result of this analysis may be a “true” or “false” output. When the goal requires the output of both the exercise and sleep queries to be true, then the goal may be satisfied only when both outputs are true. When the goal requires the output of either the exercise and sleep queries to be true, then the goal may be satisfied when at least one of the outputs are true. The evaluation may use any combination of and/or logic with one or more types of activity indicated by the biometric data.

Still referring to FIG. 2, the outputs of evaluations provided by the rule evaluator 230 may refer to rule outputs 232, which may be provided to the one or more experiment execution modules 214. The one or more experiment execution modules 214 may provide messages 234 to the one or more devices 202 that indicate the amounts of activities that a user performed, whether the amounts of activity satisfied or failed to satisfy activity goals, recommendations for activities based on whether the user has met or is on pace to meet activity goals, and the like. The one or more adherence modules 230 may provide the rule outputs 232 to one or more adherence data stores 236.

In one or more embodiments, the one or more adherence modules 227 may determine relationships between different types of biometric data. For example, the one or more adherence modules 227 may determine that a user satisfied or failed to satisfy a threshold amount of first activity and/or a threshold number of segments satisfying the threshold amount of first activity, and that a user satisfied or failed to satisfy a threshold amount of second activity and/or a threshold number of segments satisfying the threshold amount of that a user satisfied or failed to satisfy a threshold amount of first activity and/or a threshold number of segments satisfying the threshold amount of first activity. For example, the biometric data may indicate that a user has failed to meet a sleeping goal and has failed to meet an exercise goal, and the one or more adherence modules 227 may determine a correlative relationship (e.g., that not sleeping enough may have led to not exercising enough, or vice versa). In this manner, the one or more adherence modules 227 may determine an association between satisfying or failing to satisfy one or more thresholds with satisfying or failing to satisfy one or more other thresholds. The messages 234 may indicate such relationships/associations to allow a user to make behavioral decisions.

In one or more embodiments, with user consent and in compliance with any relevant laws, the network 212 may store or have access to multiple types of data (e.g., using data stores or other data storage), such as heart rate data, breathing rate data, walking data, running data, sleeping data, eating data, drinking data, and the like. A user (e.g., the user 102 of FIG. 1) may set activity goals that correspond to one or more types of data, such as running for a threshold amount of time three times a week (e.g., a first threshold number of times) for at least three weeks in a month (e.g., a second threshold number of times). In this manner, the one or more servers 140 may track segments of activity (e.g., did the user achieve a threshold number of segments of activity satisfying a threshold amount of time).

In one or more embodiments, the network 212 may, based on the goals and biometric data templates, identify the types of data to monitor. For example, the network 212 may query the one or more biometric data stores 231 for one or more types of biometric data, such as the number of running sessions, walking sessions, sleeping sessions, consumption sessions, etc. during a week. The query may specify the time range(s) for which the biometric data is desired (e.g., particular days, weeks, months, hours, etc.), and may filter biometric data returned in response to the query based on the time range(s).

In one or more embodiments, templates may define the thresholds used to monitor a person's adherence to one or more goals. Adherence rules (e.g., the adherence configuration 220) may be defined by a template. For example, and adherence rule may refer to a biometric and its corresponding threshold(s) and/or rules, which may be evaluated by a string that defines the order in which the device may evaluate the rules using Boolean operators (e.g., and, or, etc.). For example, a template may define a rule that a user must exercise a certain amount of time for a threshold number of segments in a week, and/or must sleep a threshold amount of time for a threshold number of segments in a week. The order in which the device may evaluate the relevant data to determine whether the user met a goal may be defined by the string (e.g., a computer function). In an example in which the goal is to run for at least thirty minutes and sleep between 7-9 hours every night for a week, the adherence rules may include Rule 1: A metric greater than or equal to a threshold of thirty minutes using a biometric “total running duration,” and Rule 2: A metric between thresholds of seven and nine hours of sleep using the biometric “sleep duration.” The device may fetch (e.g., request using a request call, such as a JSON) the metrics individually by querying the network biometric data storage, may receive the queried data, and may determine Boolean results (e.g., true or false) against the thresholds (e.g., true or false that a user ran for at least thirty minutes, true or false that the user slept 7-9 hours every night for a week). An aggregated rule expression may rely on one or more of the Boolean results (e.g., true and true=true—user adhered to the goal; true and false=false; user did not adhere to the goal) Likewise, the network may receive queries identifying data types, and may provide the data types to the device.

In one or more embodiments, a user's adherence may be based on units of time during which the user's adherence may be tracked (e.g., a day beginning at midnight and ending at 11:59 PM). An adherence segment may refer to a collection of adherence units of time (e.g., a segment may refer to one day or a block of hours). An adherence segment may have its own adherence value based on respective adherences of individual time units. An adherence metric may refer to a biometric used to determine the user's adherence (e.g., a step count, walking/running sessions, sleeping sessions, etc.). An adherence rule may be used to determine whether an adherence metric should count toward determining a user's adherence (e.g., whether an adherence metric was greater than a threshold, less than a threshold, between thresholds, etc.). A rule output may refer to a result of an adherence determination based on an adherence rule, and may include an indication of what the target adherence metric value was and what the user's actual value was for the adherence metric. For example, an experiment may set a goal of the user having less than eight hours of sedentary time for four weeks. The query and metric may include an aggregation query of “none” or “zero” for an activity intensity metric. The time unit rule used to evaluate the metric may be a rule of less than or equal to a threshold (e.g., zero or none) for any time units (e.g., any given day), and the segment rule may be a number of days (e.g., days during which the time unit rule was adhered to) satisfying a threshold of 28 days (e.g., four weeks). Another experiment may be to job for thirty minutes three times in a week or to run at least three times in a week. The query and metric may include a run workout session query to receive running session data. The time unit rule used to evaluate the metric may be a rule of based on the OR Boolean condition of the query, and the segment rule may be a number of days adhered (e.g., a number of days when the jogging or running was adhered to) satisfying a threshold of three days.

In one or more embodiments, the network 212 may determine whether the adherence or failure to adhere to a goal may correlate to an effect on the user's biometric data and/or the user's adherence or failure to adhere to another goal. For example, the network 212 may determine whether the user adhering to an exercise goal correlates to whether the user slept and/or ate a sufficient amount, whether the user exercising affected the user's heart rate, whether the user running a number of times affected the user's running pace, and the like. The relationships between data may be evaluated for real-time, past, and future behavioral determinations, such as whether the user may sleep better if the person adhered to an exercise goal, etc. In this manner, the network 212 may identify the impact of some biometric data on other biometric data, and may indicate the impact to the user to allow the user to make behavioral decisions. The network 212 may provide notifications and instructions (e.g., the messages 234) to the one or more devices 202 for presentation.

The one or more devices 202 may be configured to communicate via a communications network 250, and the network 212 may be configured to communicate via the communications network 260, wirelessly or wired (e.g., the same or different wireless communications networks). The communications network 250, and/or the communications network 260 may include, but not limited to, any one of a combination of different types of suitable communications networks such as, for example, broadcasting networks, cable networks, public networks (e.g., the Internet), private networks, wireless networks, cellular networks, or any other suitable private and/or public networks. Further, the communications network 250, and/or the communications network 260 may have any suitable communication range associated therewith and may include, for example, global networks (e.g., the Internet), metropolitan area networks (MANs), wide area networks (WANs), local area networks (LANs), or personal area networks (PANs). In addition, communications network 250, and/or the communications network 260 may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, white space communication mediums, ultra-high frequency communication mediums, satellite communication mediums, or any combination thereof.

The one or more devices 202 and/or the network 212 may include any suitable processor-driven device including, but not limited to, a mobile device or a non-mobile, e.g., a static, device. For example, one or more devices 202 and/or the network 212 may include a user equipment (UE), a station (STA), an access point (AP), a personal computer (PC), a wearable wireless device (e.g., bracelet, watch, glasses, ring, etc.), a desktop computer, a mobile computer, a laptop computer, an Ultrabook™ computer, a notebook computer, a tablet computer, a server computer, a handheld computer, a handheld device, an internet of things (IoT) device, a sensor device, a PDA device, a handheld PDA device, an on-board device, an off-board device, a hybrid device (e.g., combining cellular phone functionalities with PDA device functionalities), a consumer device, a vehicular device, a non-vehicular device, a mobile or portable device, a non-mobile or non-portable device, a mobile phone, a cellular telephone, a PCS device, a PDA device which incorporates a wireless communication device, a mobile or portable GPS device, a DVB device, a relatively small computing device, a non-desktop computer, a “carry small live large” (CSLL) device, an ultra mobile device (UMD), an ultra mobile PC (UMPC), a mobile internet device (MID), an “origami” device or computing device, a device that supports dynamically composable computing (DCC), a context-aware device, a video device, an audio device, an A/V device, a set-top-box (STB), a blu-ray disc (BD) player, a BD recorder, a digital video disc (DVD) player, a high definition (HD) DVD player, a DVD recorder, a HD DVD recorder, a personal video recorder (PVR), a broadcast HD receiver, a video source, an audio source, a video sink, an audio sink, a stereo tuner, a broadcast radio receiver, a flat panel display, a personal media player (PMP), a digital video camera (DVC), a digital audio player, a speaker, an audio receiver, an audio amplifier, a gaming device, a data source, a data sink, a digital still camera (DSC), a media player, a smartphone, a television, a music player, or the like. It is understood that the above is a list of devices. However, other devices, including smart devices, Internet of Things (IoT), such as lamps, climate control, car components, household components, appliances, etc. may also be included in this list.

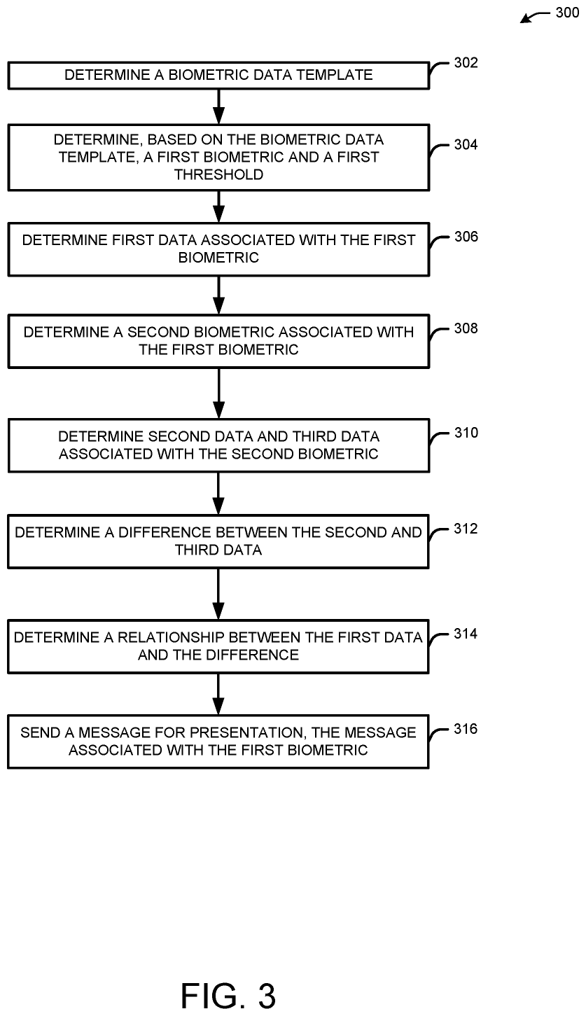

FIG. 3 illustrates an example flow diagram 300 for performing activity evaluations, in accordance with one or more example embodiments of the present disclosure.

At block 302, a device (e.g., the one or more servers 140 of FIG. 1, the network 212 of FIG. 2) may determine a biometric data template. Users may set activity goals that correspond to one or more types of data, such as running, walking, resting, sleeping, eating, drinking, and the like. The biometric data template may define one or more goals for a user. A goal may be for a user to perform an activity for amounts of time that exceed a threshold, to perform an activity for amounts of time that do not exceed a threshold, to increase or decrease an amount of activity over time, to improve performance of an activity (e.g., improve walking or running pace), to improve wellness (e.g., as measured by heart rate, breathing rate, body fat, blood glucose, etc.), and the like. The biometric data template may be selected by a user (e.g., the data 211 of FIG. 2 may indicate a selection of the biometric data template) or may be selected automatically. A goal may combine thresholds for multiple types of data across multiple units of time (e.g., using segments). For example, a goal may include a combination of sleeping time, exercise time, device usage time, and/or food or beverage consumption time, among other types of data.

At block 304, the device may determine, based on the biometric data template, a first biometric and a threshold for the first biometric. For example, the biometric data template may define the types of data to analyze and any thresholds to use in the analysis (e.g., the adherence metric query parameters 222 of FIG. 2). The types of data defined by the biometric data template may include exercise data, sleep data, consumption data, device usage data, wellness data, and the like. The biometric data template may define adherence rules, such as the time units (e.g., hours, days, weeks) to analyze, and segment rules (e.g., how many segments of the time units need to satisfy a threshold to satisfy the goal), and the like.

At block 306, the device may determine first data associated with the first biometric. The device may send a query that identifies the first biometric data defined by the biometric data template. In response, the device may receive the first biometric data. For example, when the biometric data is running data, the device may receive running segment data that indicates the days, hours, and/or weeks when the user ran, the running durations, and other relevant information indicative of the amount of running that user performed (e.g., the pace, the distance, the number of steps, etc.).

At block 308, the device may determine one or more additional biometrics associated with the first biometric. For example, the first biometric may affect multiple other biometrics, such as a second biometric and a third biometric. The first biometric may affect the second biometric independently from affecting the third biometric. For example, when the first biometric is related to consumption of food or beverage, a second biometric affected by a person's food or beverage consumption may be their exercise, and a third biometric affected by the person's food or beverage consumption may be sleep. The amount of time that a person sleeps may affect other biometrics such as exercise, health, etc. The biometric data template may define the one or more additional biometrics, or the device may determine the one or more additional biometrics to evaluate based on the first biometric data. Alternatively, based on a comparison of the first data to the first threshold, a second biometric. The device may compare, for any time unit (e.g., day, week, month, hour, etc.) whether the user performed an amount of activity associated with the first data that satisfies the threshold. For example, when the biometric data template defines a goal of running for an hour, the first data may be biometric data indicating when the user was running, and the device may analyze the first data to determine whether the running data indicates that the user ran for at least an hour in a given day, week, etc. When the biometric data template defines a goal of being sedentary less than twelve hours a day, the first data may be biometric data indicating when the user was sedentary, and the device may analyze the first data to determine whether the sedentary data indicates that the use was sedentary for less than twelve hours during any day. When a goal is satisfied or not satisfied based on exceeding or failing to exceed the threshold, the device may determine whether the success or failure may correspond to an effect on other biometric data. For example, when the first data indicates that the user did not meet a goal to reduce device screen time, the device may determine second data such as sleep data to evaluate whether the failure to reduce screen time correlates with a failure to sleep a threshold number of hours. When the first goal indicates that the user met an exercise goal, the device may determine whether the person's walking or running pace improved in comparison to a prior walking or running pace, whether the person slept more or less, whether wellness indicator (e.g., heart rate, breathing rate, etc.) improved, and the like.

At block 310, the device may determine additional (e.g., second, third, fourth, etc.) data indicating quantities of the second biometric. The device may identify the second biometric in a subsequent query for biometric data, and may receive the requested second biometric data in response. For example, second data may indicate a quantity of the second biometric at one time (e.g., one day) and third data may indicate a quantity of the second biometric at another time (e.g., another day). For example, the second data may indicate a quantity of the second biometric at a time that precedes the first data evaluated, and the third data may indicate a quantity of the second biometric at a time that overlaps the first data.

At block 312, the device may use the second and third data of the second biometric to determine a difference in the second biometric from the time of the second data to the time of the third data. A difference may indicate that satisfying or not satisfying a first goal based on the first data may correspond to the difference. For example, a person's running or walking pace may increase with more exercise or may decrease with less exercise. A person's sleeping time may change with more or less exercise. A person's exercise or sedentary time may change with more or less exercise, and the like.

At block 314, the device may identify a relationship/association between the first data and the second data. The device may use the relationship/association to indicate to the user that performance or non-performance of an activity indicated by the first data may affect the second data, or vice versa, and may predict and/or recommend performance or non-performance of the activity of the first data to the user to alter or maintain quantities of the second data. The relationship may be correlative or causal. The device may store the relationship/association data to use in determining the second data based on a success or failure indicated by the first data, and to use in making recommendations to a user or in sending instructions to control other devices.

At block 316, the device may send a message (e.g., the messages 234 of FIG. 2) to another device for presentation. The message may indicate the first data, whether the first data satisfied a goal defined by the threshold, whether the first data is related to/associated with the second data, recommendations for increasing or decreasing amounts of activity associated with the first data, and the like. The message sent to a device presentation may indicate, in real-time, the user's progress/adherence to goals. For example, when segment goals exist (e.g., adhering to a goal for a respective day multiple days during a time period), the messages may indicate whether the user 102 has adhered to or not adhered to a goal in respective segments (e.g., whether the user adhered to goal criteria for any day during a week), and/or whether the user 102 is on pace to adhere to a goal (e.g., has the user exercised enough today, slept enough today, etc.). In this manner, the message may indicate whether a user adhered to a goal over time, or whether the user is likely to adhere to a goal while the user's activities are still being evaluated for the goal.

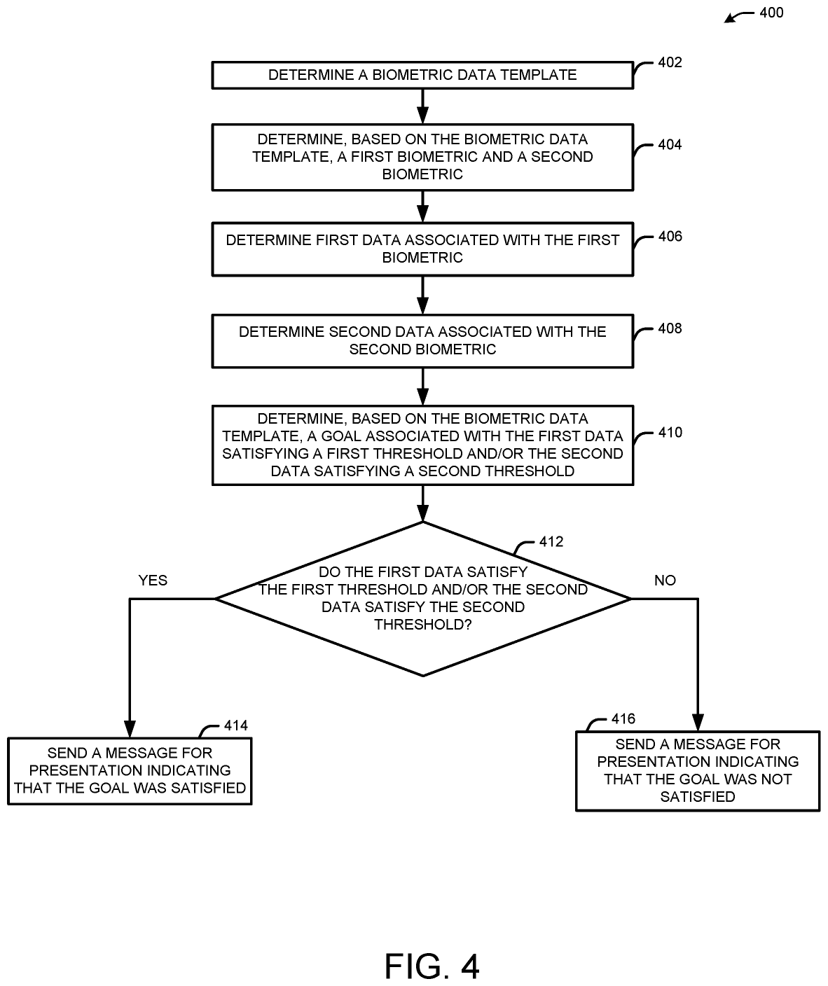

FIG. 4 illustrates a flow diagram for a process 400 for performing activity evaluations, in accordance with one or more example embodiments of the present disclosure.

At block 402, a device (e.g., the one or more servers 140 of FIG. 1, the network 212 of FIG. 2) may determine a biometric data template. Users may set activity goals that correspond to one or more types of data, such as running, walking, resting, sleeping, eating, drinking, and the like. The biometric data template may define one or more goals for a user. A goal may be for a user to perform an activity for amounts of time that exceed a threshold, to perform an activity for amounts of time that do not exceed a threshold, to increase or decrease an amount of activity over time, to improve performance of an activity (e.g., improve walking or running pace), to improve wellness (e.g., as measured by heart rate, breathing rate, body fat, blood glucose, etc.), and the like. The biometric data template may be selected by a user (e.g., the data 211 of FIG. 2 may indicate a selection of the biometric data template) or may be selected automatically. A goal may combine thresholds for multiple types of data across multiple units of time (e.g., using segments). For example, a goal may include a combination of sleeping time, exercise time, device usage time, and/or food or beverage consumption time, among other types of data.

At block 404, the device may determine, based on the biometric data template, a first biometric and a second biometric. The biometric data template may define goals that use and/or logic combinations of multiple biometrics, such as the first biometric and the second biometric. For example, a goal may require that a quantity of the first biometric satisfies a first threshold, and that a quantity of the second biometric satisfies a second threshold, or that just one of a quantity of the first biometric satisfies a first threshold or a quantity of the second biometric satisfies a second threshold. The device may send a query that identifies the first and second biometrics indicated by the biometric data template, the query requesting first data for the first biometric and second data for the second biometric.

At block 406, the device may determine the first data associated with the first biometric. At block 408, the device may determine the second data associated with the second biometric. In response to the query, the device may receive the first and second data from one or more data stores where the biometric data may be stored. For example, when the first biometric is running, the device may receive running segment data that indicates the days, hours, and/or weeks when the user ran, the running durations, and other relevant information indicative of the amount of running that user performed (e.g., the pace, the distance, the number of steps, etc.). When the second biometric is sleeping or sedentary time, the device may receive sleeping segment data or sedentary segment data that indicates the days, hours, and/or weeks when the user was sleeping or sedentary, the sleeping or sedentary durations, and the like.

At block 410, the device may determine, based on the biometric data template a goal requiring the first data to satisfy a first threshold and/or the second data to satisfy a second threshold. The biometric data template may define the rules for satisfying the goal, including the and/or logic and the order in which to evaluate the first and second data to determine respective success/failure (e.g., true/false) outputs for the first and second data, and the combined output based on the and/or logic. A goal may combine thresholds for multiple types of data across multiple units of time (e.g., using segments). For example, a goal may include a combination of sleeping time, exercise time, device usage time, and/or food or beverage consumption time, among other types of data.

At block 412, the device may determine whether the goal is satisfied based on whether the first data satisfies the first threshold and/or the second data satisfies the second threshold. The device may compare, for any time unit (e.g., day, week, month, hour, etc.) whether the user performed an amount of activity associated with the first data that satisfies the threshold. For example, when the biometric data template defines a goal of running for an hour, the first data may be biometric data indicating when the user was running, and the device may analyze the first data to determine whether the running data indicates that the user ran for at least an hour in a given day, week, etc. When the biometric data template defines a goal of being sedentary less than twelve hours a day, the first data may be biometric data indicating when the user was sedentary, and the device may analyze the first data to determine whether the sedentary data indicates that the use was sedentary for less than twelve hours during any day. The device may determine whether the first data indicates that the user performed an activity during a time segment for an amount of time that is above or below a threshold, and whether the amount of segments for which the user performed the activity above or below the threshold is above or below a threshold. When the and/or conditions defined by a rule are met, as indicated by the first data, the device may determine that first data satisfies a rule for the goal (e.g., true). The device may evaluate the second data to determine whether the second data indicates that the user performed an activity during a time segment for an amount of time that is above or below a threshold, and whether the amount of segments for which the user performed the activity above or below the threshold is above or below a threshold. When the and/or conditions defined by a rule are met, as indicated by the second data, the device may determine that second data satisfies a rule for the goal (e.g., true). Based on the individual true/false determinations for the first and second data, the device may determine whether the goal is satisfied based on the and/or rules of the goal. When the goal is satisfied, the process 400 may continue at block 414. When the goal has not been satisfied, the process 400 may continue to block 416.

At block 414, the device may send a message (e.g., the messages 234 of FIG. 2) for presentation to another device, the message indicating that the goal was satisfied (e.g., as shown in FIG. 1). At block 416, the device may send a message to another device indicating that the goal was not satisfied. The messages may indicate the first data, whether the first data satisfied a goal defined by the threshold, whether the first data is related to/associated with the second data, recommendations for increasing or decreasing amounts of activities associated with the first data or the second data, and the like.

In one or more embodiments, the message sent to a device presentation (e.g., at block 414 or block 416) may indicate, in real-time, the user's progress/adherence to goals. For example, when segment goals exist (e.g., adhering to a goal for a respective day multiple days during a time period), the messages may indicate whether the user 102 has adhered to or not adhered to a goal in respective segments (e.g., whether the user adhered to goal criteria for any day during a week), and/or whether the user 102 is on pace to adhere to a goal (e.g., has the user exercised enough today, slept enough today, etc.). In this manner, the message may indicate whether a user adhered to a goal over time, or whether the user is likely to adhere to a goal while the user's activities are still being evaluated for the goal. Block 412 may determine whether the user has satisfied a threshold for a particular segment (e.g., today, yesterday, etc.) that is associated with the evaluation of a goal that considers multiple segments, such as whether the user has exercised a threshold amount of time in a day for a threshold number of days.

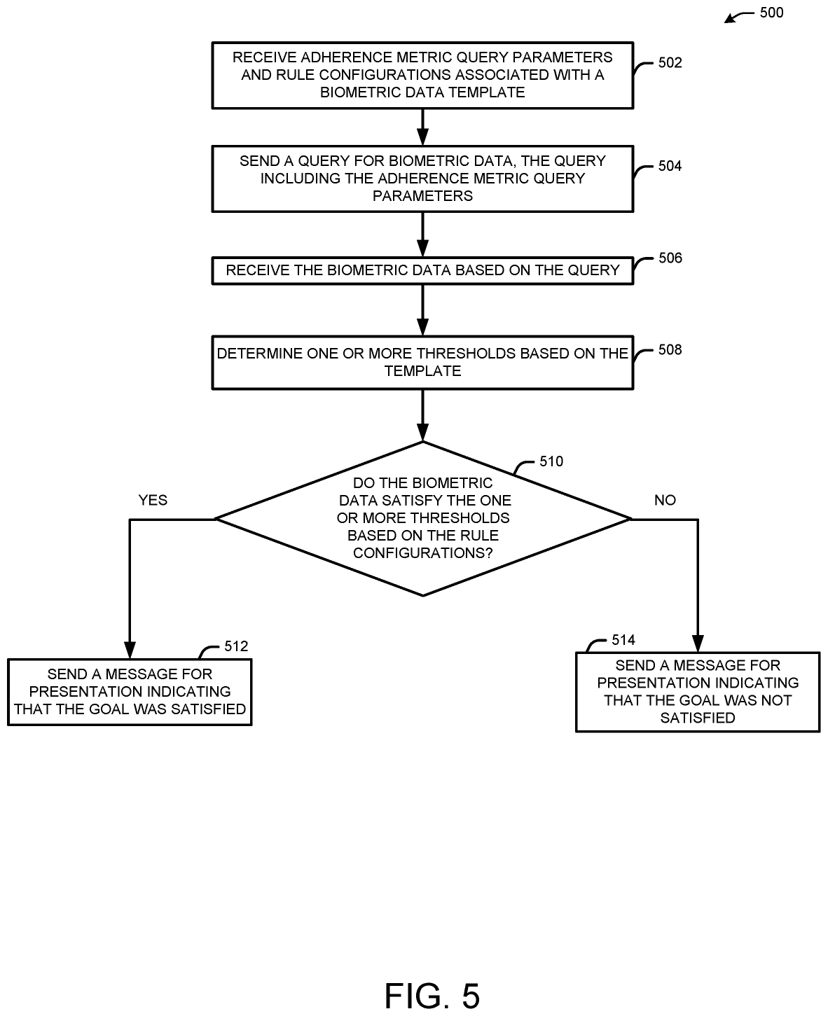

FIG. 5 illustrates a flow diagram for a process 500 for performing activity evaluations, in accordance with one or more example embodiments of the present disclosure.

At block 502, a device (e.g., the one or more servers 140 of FIG. 1, the network 212 of FIG. 2) may receive adherence metric query parameters (e.g., the adherence metric query parameters 222 of FIG. 2) and rule configurations (e.g., the time rule configuration 224 of FIG. 2, the segment rule configuration 226 of FIG. 2) associated with a biometric data template. Users may set activity goals that correspond to one or more types of data, such as running, walking, resting, sleeping, eating, drinking, and the like. The biometric data template may define one or more goals for a user. A goal may be for a user to perform an activity for amounts of time that exceed a threshold, to perform an activity for amounts of time that do not exceed a threshold, to increase or decrease an amount of activity over time, to improve performance of an activity (e.g., improve walking or running pace), to improve wellness (e.g., as measured by heart rate, breathing rate, body fat, blood glucose, etc.), and the like. The biometric data template may be selected by a user (e.g., the data 211 of FIG. 2 may indicate a selection of the activity template) or may be selected automatically. A goal may combine thresholds for multiple types of data across multiple units of time (e.g., using segments). For example, a goal may include a combination of sleeping time, exercise time, device usage time, and/or food or beverage consumption time, among other types of data. The adherence metric query parameters may include tracked biometric data (with user consent, such as step count, walk/run sessions, etc.). The rule configurations 224 indicate a threshold amount of time for which a user's activity is to satisfy a threshold (e.g., when a goal is to run for thirty minutes, the threshold may be thirty minutes), and/or may indicate a threshold number of days for which the threshold amount of time is to be satisfied.

At block 504, the device may send a query for biometric data, the query including the adherence metric query parameters. Because the biometric data template may define the adherence metric query parameters, the query may identify the adherence metric query parameters so that the biometric data provided in response to the query correspond to the relevant biometric data for the device to analyze for adherence to the biometric data template goals.

At block 506, the device may receive the biometric data based on the query. In response to the query, the device may receive the biometric data from one or more data stores where the biometric data may be stored. For example, when the biometric is running, the device may receive running segment data that indicates the days, hours, and/or weeks when the user ran, the running durations, and other relevant information indicative of the amount of running that user performed (e.g., the pace, the distance, the number of steps, etc.). When the biometric is sleeping or sedentary time, the device may receive sleeping segment data or sedentary segment data that indicates the days, hours, and/or weeks when the user was sleeping or sedentary, the sleeping or sedentary durations, and the like.

At block 508, device may determine one or more thresholds, defined by the biometric data template, for use in evaluating the biometric data to determine whether the biometric data indicates that a person adhered to the rules defined by the biometric data template (e.g., satisfying one or more goals). For example, the biometric data template may define the types of data to analyze and any thresholds to use in the analysis (e.g., the adherence metric query parameters 222 of FIG. 2). The types of data defined by the biometric data template may include exercise data, sleep data, consumption data, device usage data, wellness data, and the like. The biometric data template may define adherence rules, such as the time units (e.g., hours, days, weeks) to analyze, and segment rules (e.g., how many segments of the time units need to satisfy a threshold to satisfy the goal), and the like.

At block 510, the device may determine whether the biometric data satisfy the one or more thresholds based on the rule configurations defined by the biometric data template. The device may compare, for any time unit (e.g., day, week, month, hour, etc.) whether the user performed an amount of activity associated with the first data that satisfies the threshold. For example, when the biometric data template defines a goal of running for an hour, the biometric data may be biometric data indicating when the user was running, and the device may analyze the biometric data to determine whether the running data indicates that the user ran for at least an hour in a given day, week, etc. When the biometric data template defines a goal of being sedentary less than twelve hours a day, the biometric data may be biometric data indicating when the user was sedentary, and the device may analyze the biometric data to determine whether the sedentary data indicates that the use was sedentary for less than twelve hours during any day. The device may determine whether the biometric data indicates that the user performed an activity during a time segment for an amount of time that is above or below a threshold, and whether the amount of segments for which the user performed the activity above or below the threshold is above or below a threshold. When the and/or conditions defined by a rule are met, as indicated by the first data, the device may determine that biometric data satisfies a rule for the goal (e.g., true). The device may evaluate the biometric data to determine whether the second data indicates that the user performed an activity during a time segment for an amount of time that is above or below a threshold, and whether the amount of segments for which the user performed the activity above or below the threshold is above or below a threshold. When the and/or conditions defined by a rule are met, as indicated by the biometric data, the device may determine that biometric data satisfies a rule for the goal (e.g., true). When the goal is satisfied, the process 500 may continue at block 512. When the goal has not been satisfied, the process 500 may continue to block 514.

At block 512, the device may send a message (e.g., the messages 234 of FIG. 2) for presentation to another device, the message indicating that the goal was satisfied (e.g., as shown in FIG. 1). At block 514, the device may send a message to another device indicating that the goal was not satisfied. The messages may indicate the first data, whether the first data satisfied a goal defined by the threshold, whether the first data is related to/associated with the second data, recommendations for increasing or decreasing amounts of activities associated with the first data or the second data, and the like.

The descriptions herein are not meant to be limiting.

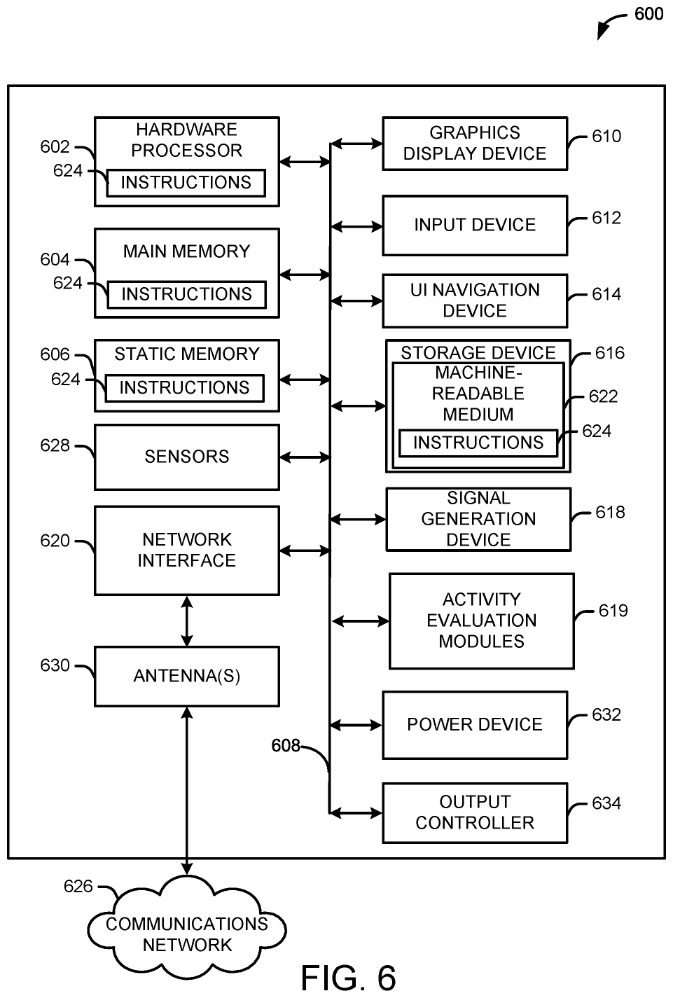

FIG. 6 illustrates a block diagram of an example of a machine 600 (e.g., the device 104 of FIG. 1, the device 106 of FIG. 1, the device 108 of FIG. 1, the one or more devices 202 of FIG. 2, the network 212 of FIG. 2) or system upon which any one or more of the techniques (e.g., methodologies) discussed herein may be performed. In other embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in Wi-Fi direct, peer-to-peer (P2P), cellular, (or other distributed) network environments. The machine 600 may be a server, a personal computer (PC), a smart home device, a tablet PC, a set-top box (STB), a personal digital assistant (PDA), a mobile telephone, a wearable computer device, a web appliance, a network router, a switch or bridge, or any machine capable of executing instructions (sequential or otherwise) that specify actions to be taken by that machine, such as a base station. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), or other computer cluster configurations.

Examples, as described herein, may include or may operate on logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In another example, the hardware may include configurable execution units (e.g., transistors, circuits, etc.) and a computer readable medium containing instructions where the instructions configure the execution units to carry out a specific operation when in operation. The configuring may occur under the direction of the executions units or a loading mechanism. Accordingly, the execution units are communicatively coupled to the computer-readable medium when the device is operating. In this example, the execution units may be a member of more than one module. For example, under operation, the execution units may be configured by a first set of instructions to implement a first module at one point in time and reconfigured by a second set of instructions to implement a second module at a second point in time.

The machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a power management device 632, a graphics display device 610, an alphanumeric input device 612 (e.g., a keyboard), and a user interface (UI) navigation device 614 (e.g., a mouse). In an example, the graphics display device 610, alphanumeric input device 612, and UI navigation device 614 may be a touch screen display. The machine 600 may additionally include a storage device (i.e., drive unit) 616, a signal generation device 618, one or more activity evaluation modules 619 (e.g., capable of performing steps according to the blocks of FIGS. 3-5), a network interface device/transceiver 620 coupled to antenna(s) 630, and one or more sensors 628, such as a biometric sensor, a global positioning system (GPS) sensor, a compass, an accelerometer, or other biometric and/or motion sensor. The machine 600 may include an output controller 634, such as a serial (e.g., universal serial bus (USB), parallel, or other wired or wireless (e.g., infrared (IR), near field communication (NFC), etc.) connection to communicate with or control one or more peripheral devices (e.g., a printer, a card reader, etc.)).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within the static memory 606, or within the hardware processor 602 during execution thereof by the machine 600. In an example, one or any combination of the hardware processor 602, the main memory 604, the static memory 606, or the storage device 616 may constitute machine-readable media.

While the machine-readable medium 622 is illustrated as a single medium, the term “machine-readable medium” may include a single medium or multiple media (e.g., a centralized or distributed database, and/or associated caches and servers) configured to store the one or more instructions 624.

Various embodiments may be implemented fully or partially in software and/or firmware. This software and/or firmware may take the form of instructions contained in or on a non-transitory computer-readable storage medium. Those instructions may then be read and executed by one or more processors to enable performance of the operations described herein. The instructions may be in any suitable form, such as but not limited to source code, compiled code, interpreted code, executable code, static code, dynamic code, and the like. Such a computer-readable medium may include any tangible non-transitory medium for storing information in a form readable by one or more computers, such as but not limited to read only memory (ROM); random access memory (RAM); magnetic disk storage media; optical storage media; a flash memory, etc.

The term “machine-readable medium” may include any medium that is capable of storing, encoding, or carrying instructions for execution by the machine 600 and that cause the machine 600 to perform any one or more of the techniques of the present disclosure, or that is capable of storing, encoding, or carrying data structures used by or associated with such instructions. Non-limiting machine-readable medium examples may include solid-state memories and optical and magnetic media. In an example, a massed machine-readable medium includes a machine-readable medium with a plurality of particles having resting mass. Specific examples of massed machine-readable media may include non-volatile memory, such as semiconductor memory devices (e.g., electrically programmable read-only memory (EPROM), or electrically erasable programmable read-only memory (EEPROM)) and flash memory devices; magnetic disks, such as internal hard disks and removable disks; magneto-optical disks; and CD-ROM and DVD-ROM disks.

The instructions 624 may further be transmitted or received over a communications network 626 using a transmission medium via the network interface device/transceiver 620 utilizing any one of a number of transfer protocols (e.g., frame relay, internet protocol (IP), transmission control protocol (TCP), user datagram protocol (UDP), hypertext transfer protocol (HTTP), etc.). Example communications networks may include a local area network (LAN), a wide area network (WAN), a packet data network (e.g., the Internet), mobile telephone networks (e.g., cellular networks), plain old telephone (POTS) networks, wireless data networks (e.g., Institute of Electrical and Electronics Engineers (IEEE) 602.11 family of standards known as Wi-Fi®, IEEE 602.16 family of standards known as WiMax®), IEEE 602.15.4 family of standards, and peer-to-peer (P2P) networks, among others. In an example, the network interface device/transceiver 620 may include one or more physical jacks (e.g., Ethernet, coaxial, or phone jacks) or one or more antennas to connect to the communications network 626. In an example, the network interface device/transceiver 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600 and includes digital or analog communications signals or other intangible media to facilitate communication of such software.

The operations and processes described and shown above may be carried out or performed in any suitable order as desired in various implementations. Additionally, in certain implementations, at least a portion of the operations may be carried out in parallel. Furthermore, in certain implementations, less than or more than the operations described may be performed.

The word “exemplary” is used herein to mean “serving as an example, instance, or illustration.” Any embodiment described herein as “exemplary” is not necessarily to be construed as preferred or advantageous over other embodiments. The terms “computing device,” “user device,” “communication station,” “station,” “handheld device,” “mobile device,” “wireless device” and “user equipment” (UE) as used herein refers to a wireless communication device such as a cellular telephone, a smartphone, a tablet, a netbook, a wireless terminal, a laptop computer, a femtocell, a high data rate (HDR) subscriber station, an access point, a printer, a point of sale device, an access terminal, or other personal communication system (PCS) device. The device may be either mobile or stationary.

As used within this document, the term “communicate” is intended to include transmitting, or receiving, or both transmitting and receiving. This may be particularly useful in claims when describing the organization of data that is being transmitted by one device and received by another, but only the functionality of one of those devices is required to infringe the claim. Similarly, the bidirectional exchange of data between two devices (both devices transmit and receive during the exchange) may be described as “communicating,” when only the functionality of one of those devices is being claimed. The term “communicating” as used herein with respect to a wireless communication signal includes transmitting the wireless communication signal and/or receiving the wireless communication signal. For example, a wireless communication unit, which is capable of communicating a wireless communication signal, may include a wireless transmitter to transmit the wireless communication signal to at least one other wireless communication unit, and/or a wireless communication receiver to receive the wireless communication signal from at least one other wireless communication unit.

As used herein, unless otherwise specified, the use of the ordinal adjectives “first,” “second,” “third,” etc., to describe a common object, merely indicates that different instances of like objects are being referred to and are not intended to imply that the objects so described must be in a given sequence, either temporally, spatially, in ranking, or in any other manner.

Some embodiments may be used in conjunction with various devices and systems, for example, a personal computer (PC), a desktop computer, a mobile computer, a laptop computer, a notebook computer, a tablet computer, a server computer, a handheld computer, a handheld device, a personal digital assistant (PDA) device, a handheld PDA device, an on-board device, an off-board device, a hybrid device, a vehicular device, a non-vehicular device, a mobile or portable device, a consumer device, a non-mobile or non-portable device, a wireless communication station, a wireless communication device, a wireless access point (AP), a wired or wireless router, a wired or wireless modem, a video device, an audio device, an audio-video (A/V) device, a wired or wireless network, a wireless area network, a wireless video area network (WVAN), a local area network (LAN), a wireless LAN (WLAN), a personal area network (PAN), a wireless PAN (WPAN), and the like.

Some embodiments may be used in conjunction with one way and/or two-way radio communication systems, cellular radio-telephone communication systems, a mobile phone, a cellular telephone, a wireless telephone, a personal communication system (PCS) device, a PDA device which incorporates a wireless communication device, a mobile or portable global positioning system (GPS) device, a device which incorporates a GPS receiver or transceiver or chip, a device which incorporates an RFID element or chip, a multiple input multiple output (MIMO) transceiver or device, a single input multiple output (SIMO) transceiver or device, a multiple input single output (MIS 0) transceiver or device, a device having one or more internal antennas and/or external antennas, digital video broadcast (DVB) devices or systems, multi-standard radio devices or systems, a wired or wireless handheld device, e.g., a smartphone, a wireless application protocol (WAP) device, or the like.

Some embodiments may be used in conjunction with one or more types of wireless communication signals and/or systems following one or more wireless communication protocols, for example, radio frequency (RF), infrared (IR), frequency-division multiplexing (FDM), orthogonal FDM (OFDM), time-division multiplexing (TDM), time-division multiple access (TDMA), extended TDMA (E-TDMA), general packet radio service (GPRS), extended GPRS, code-division multiple access (CDMA), wideband CDMA (WCDMA), CDMA 2000, single-carrier CDMA, multi-carrier CDMA, multi-carrier modulation (MDM), discrete multi-tone (DMT), Bluetooth®, global positioning system (GPS), Wi-Fi, Wi-Max, ZigBee, ultra-wideband (UWB), global system for mobile communications (GSM), 2G, 2.5G, 3G, 3.5G, 4G, fifth generation (5G) mobile networks, 3GPP, long term evolution (LTE), LTE advanced, enhanced data rates for GSM Evolution (EDGE), or the like. Other embodiments may be used in various other devices, systems, and/or networks.

It is understood that the above descriptions are for purposes of illustration and are not meant to be limiting.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Program module(s), applications, or the like disclosed herein may include one or more software components including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform.

Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component comprising instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in any applicable flow diagrams to be performed. These computer program instructions may also be stored in a computer-readable storage medium (CRSM) that upon execution may direct a computer or other programmable data processing apparatus to function in a particular manner, such that the instructions stored in the computer-readable storage medium produce an article of manufacture including instruction means that implement one or more functions or operations specified in any flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

What is claimed is:

1. A method, comprising:
determining, by at least one processor of a phone device, an activity template;
determining, by the at least one processor and based on the activity template, heart rate data associated with exercise;
determining, by the at least one processor, that the heart rate data is indicative of a quantity of exercise on a first day;
determining, by the at least one processor, breathing data associated with sleeping;
determining, by the at least one processor, that the breathing data is indicative of a first quantity of sleep at a first time and indicative of a second quantity of sleep at a second time;
determining, by the at least one processor, a difference between the first quantity of sleep and the second quantity of sleep;
determining, by the at least one processor, that the difference between the first quantity of sleep and the second quantity of sleep is based on the quantity of exercise; and
presenting, by the at least one processor, a message associated with the quantity of exercise and the difference between the first quantity of sleep and the second quantity of sleep.

2. The method of claim 1, wherein:
the first time is prior to the first day and the second time is after the first time, further comprising:
determining second heart rate data indicative of a second quantity of exercise at a third time before the first time; and
comparing the second quantity of exercise to a threshold quantity of exercise,
wherein determining that the difference is based on the quantity of exercise is further based on the comparison of the second quantity of exercise to the threshold quantity of exercise.

3. The method of claim 1, further comprising:
determining, based on the activity template, a threshold number of days associated with exercise, the threshold number of days greater than one;
determining that the heart rate data is further indicative of a second quantity of exercise on a second day and a third quantity of exercise on a third day;
determining that the quantity of exercise, the second quantity of exercise, and the third quantity of exercise satisfy a threshold quantity of exercise;
determining, based on the first day, the second day, and the third day, a number of days that the heart rate data indicate quantities of exercise that satisfy the threshold quantity of exercise; and
determining that the number of days exceeds the threshold number of days,
wherein the message indicates that a goal associated with the threshold number of days has been satisfied.

4. The method of claim 1, further comprising:
determining, based on the activity template, a first threshold number of hours associated with exercise and a second threshold number of hours associated with sleeping;
determining, based on the first day, a first number of days that the heart rate data indicate quantities of exercise that satisfy the first threshold number of hours;
determining, based on the first time and the second time, a second number of days that the breathing data indicate quantities of sleep that satisfy the second threshold number of hours;
determining that the first number of days exceeds the first threshold number of days; and
determining that the second number of days exceeds the second threshold number of days,
wherein the message indicates that a goal associated with the first threshold number of days and the second threshold number of days has been satisfied.

5. A method, comprising:
determining by at least one processor of a first device and based on an activity template, first biometric data;
determining, by the at least one processor, that the first biometric data is indicative of a first quantity of a first activity;
determining, by the at least one processor and based on the first quantity of the first activity, second biometric data indicative of a second quantity of a second activity, the first activity different than the second activity;
determining, by the at least one processor, that the second quantity of the second activity is based on the first quantity of the first activity; and
causing presentation, by the at least one processor, of a message associated with the first biometric data.

6. The method of claim 5, further comprising:
determining that the first quantity of the first activity exceeds a threshold quantity of the first activity;
determining a threshold quantity of the second activity; and
determining, based on the second biometric data, that the second quantity of the second activity exceeds the threshold quantity of the second activity,
wherein determining that the second quantity of the second activity is based on the first quantity of the first activity comprises determining that the first quantity of the first activity exceeding the threshold quantity of the first activity is indicative of the second quantity of the second activity exceeding the threshold quantity of the second activity.

7. The method of claim 5, further comprising:
determining that the first quantity of the first activity fails to exceed a threshold quantity of the first activity;
determining a threshold quantity of the second activity; and
determining, based on the second biometric data, that the second quantity of the second activity fails to exceed the threshold quantity of the second activity,
wherein determining that the second quantity of the second activity is based on the first quantity of the first activity comprises determining that the first quantity of the first activity failing to exceed the threshold quantity of the first activity is indicative of the second quantity of the second activity failing to exceed the threshold quantity of the second activity.

8. The method of claim 5, the second biometric data further indicative of a third quantity of the second activity, the method further comprising:
determining a difference between the second quantity of the second activity and the third quantity of the second activity; and
determining that the first quantity of the first activity exceeds a threshold quantity of the first activity,
wherein determining that the second quantity of the second activity is based on the first quantity of the first activity comprises determining that the first quantity of the first activity exceeding the threshold quantity of the first activity is indicative of the difference.

9. The method of claim 5, the second biometric data further indicative of a third quantity of the second activity, further comprising:
determining a difference between the second quantity of the second activity and the third quantity of the second activity; and
determining that the first quantity of the first activity fails to exceed a threshold quantity of the first activity, wherein:
determining that the second quantity of the second activity is based on the first quantity of the first activity comprises determining that the first quantity failing to exceed the threshold quantity of the first activity is indicative of the difference, and
the first quantity is a quantity of the first activity on a first day, the second quantity is a quantity of the second activity on the first day and the third quantity is a quantity of the second activity on a second day.

10. The method of claim 5, wherein:
the first biometric data is associated with a first time,
the second biometric data is associated with a second time prior to the first time, the method further comprising:
determining third biometric data associated with the first activity and a third time before the first time; and
comparing the third biometric data to a threshold quantity of the first activity,
wherein determining that the second quantity of the second activity is based on the first quantity of the first activity is based on the comparison of the third biometric data to the threshold quantity of the first activity.

11. The method of claim 5, the first quantity being a first quantity of the first activity on a first day, the method further comprising:
determining, based on the activity template, a threshold number of days associated with the first activity, the threshold number of days greater than one;
determining that the first biometric data is further indicative of a third quantity of the first activity on a second day and a fourth quantity of the first activity on a third day;
determining that the first quantity, the third quantity, and the fourth quantity satisfy a threshold number of hours of the first activity;
determining, based on the first day, the second day, and the third day, a number of days that the first biometric data indicate quantities of the first activity that satisfy the threshold number of hours of the first activity; and
determining that the number of days exceeds the threshold number of days,
wherein the message indicates that a goal associated with the threshold number of days has been satisfied.

12. The method of claim 5, the first quantity being a first quantity of the first activity on a first day, the method further comprising:
determining, based on the activity template, a threshold number of days associated with the first activity, the threshold number of days greater than one;
determining that the first biometric data is further indicative of a third quantity of the first activity on a second day and a fourth quantity of the first activity on a third day;
determining that the first quantity, the third quantity, and the fourth quantity satisfy a threshold number of hours of the first activity;
determining, based on the first day, the second day, and the third day, a number of days that the first biometric data indicate quantities of the first activity that satisfy the threshold number of hours of the first activity; and
determining that the number of days fails to exceed the threshold number of days,
wherein the message indicates that a goal associated with the threshold number of days has not been satisfied.

13. The method of claim 5, the first quantity being a first quantity of the first activity on a first day, the method further comprising:
determining, based on the activity template, a first threshold number of days associated with the first activity and a second threshold number of days associated with the second activity;
determining, based on the first day, a first number of days that the first biometric data indicate quantities of the first activity that satisfy the first threshold number of days;
determining a second number of days that the second biometric data indicate quantities of the second activity that satisfy the second threshold number of days;
determining that the first number of days exceeds the first threshold number of days; and
determining that the second number of days exceeds the second threshold number of days,
wherein the message indicates that a goal associated with the first threshold number of days and the second threshold number of days has been satisfied.

14. The method of claim 5, the first quantity being a first quantity of the first activity on a first day, the method further comprising:
determining, based on the activity template, a first threshold number of days associated with the first activity and a second threshold number of days associated with the second activity;
determining, based on the first day, a first number of days that the first biometric data indicate quantities of the first activity that satisfy the first threshold number of days;
determining a second number of days that the second biometric data indicate quantities of the second activity that satisfy the second threshold number of days;
determining that the first number of days exceeds the first threshold number of days; and
determining that the second number of days fails to exceed the second threshold number of days;
wherein the message indicates that a goal associated with the first threshold number of days and the second threshold number of days has not been satisfied.

15. The method of claim 5, further comprising:
receiving parameters associated with the activity template;
sending a request for the parameters; and
receiving, based on the request, the first biometric data and the second biometric data.

16. The method of claim 5, wherein the message indicates that the second quantity is based on the first quantity.

17. A system comprising memory coupled to at least one processor, the at least one processor configured to:
determine, based on an activity template, first biometric data and a first threshold amount of activity associated with the first biometric data;
determine that the first biometric data is indicative of a first quantity of a first activity;
determine, based on the activity template, a first threshold number of segments associated with the first activity;
determine a first number of segments during which the first biometric data indicate quantities of the first activity that satisfy the first threshold amount of activity;
determine that the first number of segments exceeds the first threshold number of segments; and
send a message to a device for presentation, the message indicating that a goal associated with the first threshold number of segments has been satisfied.

18. The system of claim 17, wherein the at least one processor is further configured to:
determine second biometric data and a second threshold amount of activity associated with the second biometric data;
determine that the second biometric data is indicative of a second quantity of a second activity;
determine a second threshold number of segments associated with the second activity;
determine a second number of segments during which the second biometric data indicate quantities of the second activity that satisfy the second threshold amount of activity; and
determine that the second number of segments exceeds the second threshold number of segments,
wherein the message further indicates that the goal is associated with the first threshold number of segments and the second threshold number of segments.

19. The system of claim 17, wherein the first biometric data indicates that the first quantity of the first activity occurred during a first time period, and wherein the at least one processor is further configured to:
determine that the first biometric data is further indicative of a second quantity of the first activity during a second time period after the first time period and a third quantity of the first activity during a third time period after the second time period; and
determine that the second quantity of the first activity and the third quantity of the first activity satisfy the first threshold amount of activity,
wherein to determine that the first number of segments during which the first biometric data indicate quantities of the first activity that satisfy the first threshold amount of activity is further based on the second time period and the third time period.

20. The system of claim 17, wherein the at least one processor is further configured to:
receive parameters associated with the activity template;
send a request comprising the parameters; and
receive, based on the request, the first biometric data and the second biometric data.
